(19) United States
(12) Patent Application Publication
(10) Pub. No.: US 2012/0232338 A1
(43) Pub. Date: Sep. 13, 2012

(54) SURGICAL INSTRUMENT WITH DETACHABLE TIP

(76) Inventor: Steve Livneh, Amherstburg (CA)

(21) Appl. No.: 13/372,247

(22) Filed: Feb. 13, 2012

Related U.S. Application Data
(60) Provisional application No. 61/441,971, filed on Feb. 11, 2011.

(51) Int. Cl. A61B 17/94; A61B 1/00

(52) U.S. Cl. 600/104

(57) ABSTRACT

An endoscopic hand instrument includes a tubular member having a throughbore extending between a first open end and a second open end, the throughbore including a first portion having a first inner diameter and a second portion having a second inner diameter, a collet member received within the throughbore, the collet member including at least two biasable arm members, the collet member being biased such that the two arm members extend radially outward into the first portion of the throughbore, the two arm members including an area defining an aperture, and a tip assembly having an engagement member, the engagement member being received between the two arm members and into the aperture, the tip assembly engaging the tubular member so as to force the two arm members into the second portion of the throughbore such that the two arm members are prevented from extending radially outward.

### CROSS-REFERENCE TO RELATED APPLICATION

The instant application claims priority to U.S. Provisional Patent Application Ser. No. 61/441,971, filed Feb. 11, 2011, pending, the entire specification of which is expressly incorporated herein by reference.

### FIELD OF INVENTION

The present invention relates generally to surgical instruments, and more particularly, to devices and systems to attach and detach jaw tips to surgical hand instruments, especially those jaw tips that are at least partially disposable and/or reposable.

### BACKGROUND OF INVENTION

General surgery, endoscopy and especially laparoscopic endoscopy have been rapidly growing practices in the past decades. Currently, these types of surgical procedures are generally performed mostly with hand held instruments, with various jaw configurations such as scissors, graspers, dissectors and/or the like that are operably associated therewith.

There is a growing trend to reduce the amount of waste and contamination by reducing the volume of disposable and contaminated surgical waste. The use of reposable and partially disposable devices, especially various jaw tips, is growing steadily as a result. However, conventional attempts to reduce waste and contamination have not been entirely satisfactory. For example, conventional disposable and/or reposable jaw tips are difficult to securely engage to the shaft and/or tube/handle portions of the hand held instruments. This can lead to inadvertent or unintended separation of the jaw tip from the shaft and/or tube/handle portions of the hand held instruments either before and/or during the surgical procedure. This occurrence, apart from endangering the safety of the patient, would also necessitate the use of another jaw tip, thus leading to even more surgical waste and contamination concerns.

Accordingly, there exists a need for new and improved surgical devices and systems that can overcome at least one of the afore-mentioned shortcomings.

### SUMMARY OF THE INVENTION

In accordance with the general teachings of the present invention, new and improved surgical instruments with attachable and detachable jaw tips are provided.

More specifically, the present invention provides an efficient and simple way to attach jaws tips to surgical devices and allows for easy attachment and detachment and thus allows for the disposal of worn-out tips while preserving the reusable shaft and/or tube/handle portions. Adopting these designs are intended to allow easier and safer use of disposable and reposable jaw tips.

In accordance with a first embodiment of the present invention, an endoscopic hand instrument, comprising: a tubular member having an area defining a throughbore extending between a first open end and a second open end, wherein the throughbore includes a first portion having a first inner diameter and a second portion having a second inner diameter, wherein the first inner diameter is greater than the second inner diameter; a collet member selectively operable to be at least partially received within the throughbore, wherein the collet member includes at least two biasable arm members, wherein the collet member is biased such that the two arm members extend radially outward into the first portion of the throughbore, wherein the two arm members include an area defining an aperture; and a tip assembly having an engagement member, wherein the engagement member is selectively operable to be received between the two arm members and into the aperture, wherein the tip assembly is selectively operable to engage the tubular member so as to force the two arm members into the second portion of the throughbore such that the two arm members are prevented from extending radially outward whereby the engagement member is secured in the aperture.

In accordance with a second embodiment of the present invention, an endoscopic hand instrument, comprising: a tubular member having an area defining a throughbore extending between a first open end and a second open end, wherein the throughbore includes a first portion having a first inner diameter and a second portion having a second inner diameter, wherein the first inner diameter is greater than the second inner diameter; an actuation shaft member selectively operable to be at least partially received within the throughbore, wherein the actuation shaft member is selectively operable to more in a to and fro direction within the throughbore; a collet member operably associated with an end portion of the actuation shaft member, wherein the collet member is selectively operable to be at least partially received within the throughbore, wherein the collet member includes at least two biasable arm members, wherein the collet member is biased such that the two arm members extend radially outward into the first portion of the throughbore, wherein the two arm members include an area defining an aperture; and a tip assembly having an engagement member, wherein the engagement member is selectively operable to be received between the two arm members and into the aperture, wherein the tip assembly is selectively operable to engage the tubular member so as to force the two arm members into the second portion of the throughbore such that the two arm members are prevented from extending radially outward whereby the engagement member is secured in the aperture.

In accordance with a third embodiment of the present invention, an endoscopic hand instrument, comprising: a tubular member having an area defining a throughbore extending between a first open end and a second open end, wherein the throughbore includes a first portion having a first inner diameter and a second portion having a second inner diameter, wherein the first inner diameter is greater than the second inner diameter; a handle assembly operably associated with the tubular member, wherein the handle assembly includes at least one movable handle member; an actuation shaft member selectively operable to be at least partially received within the throughbore, wherein at least one end of the actuation shaft member is operably associated with the movable handle member, wherein the actuation shaft member is selectively operable to more in a to and fro direction within the throughbore; a collet member operably associated with an end portion of the actuation shaft member, wherein the collet member is selectively operable to be at least partially received within the throughbore, wherein the collet member includes at least two biasable arm members, wherein the collet member is biased such that the two arm members extend radially outward into the first portion of the throughbore, wherein the two arm members include an area defining an aperture; and a tip assembly having an engagement member, wherein the engagement member is selectively operable to be received between the two arm members and into the aperture, wherein the tip assembly is selectively operable to engage the tubular member so as to force the two arm members into the second portion of the throughbore such that the two arm members are prevented from extending radially outward whereby the engagement member is secured in the aperture.

Further areas of applicability of the present invention will become apparent from the detailed description provided hereinafter. It should be understood that the detailed description and specific examples, while indicating the preferred embodiment of the invention, are intended for purposed of illustration only and are not intended to limit the scope of the invention.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 illustrates an elevational view of an endoscopic hand instrument, in accordance with a first embodiment of the present invention;

FIG. 2 illustrates an exploded view of an endoscopic hand instrument, in accordance with a second embodiment of the present invention;

FIG. 3 illustrates a partial sectional view of a distal portion of a tube member of an endoscopic hand instrument, in accordance with a third embodiment of the present invention;

FIG. 4 illustrates a perspective view of a bushing member of an endoscopic hand instrument, in accordance with a fourth embodiment of the present invention;

FIG. 5 illustrates a partial sectional view of a knob member/tube member/handle system assembly of an endoscopic hand instrument, in accordance with a fifth embodiment of the present invention;

FIG. 6 illustrates an elevational view of a collet member of an endoscopic hand instrument, in accordance with a sixth embodiment of the present invention;

FIG. 7 illustrates a sectional view taken along line I-I of FIG. 6, in accordance with a seventh embodiment of the present invention;

FIG. 8 illustrates a perspective view of an endoscopic hand instrument wherein a collet member is received into the distal portion of a tube member, in accordance with an eighth embodiment of the present invention;

FIG. 9 illustrates a partial sectional view of an endoscopic hand instrument wherein a collet member is received into the distal portion of a tube member, in accordance with a ninth embodiment of the present invention;

FIG. 10 illustrates a perspective view of a tip system of an endoscopic hand instrument, in accordance with a tenth embodiment of the present invention;

FIG. 11 illustrates a perspective view of an alternative tip system of an endoscopic hand instrument, in accordance with an eleventh embodiment of the present invention;

FIG. 12 illustrates an exploded view of a tip system of an endoscopic hand instrument, in accordance with a twelfth embodiment of the present invention;

FIG. 12a illustrates a perspective view of a clevis member of a tip system of an endoscopic hand instrument, in accordance with a thirteenth embodiment of the present invention;

FIG. 13 illustrates a partial sectional view of a tip system engaged to a collet member of an endoscopic hand instrument, in accordance with a fourteenth embodiment of the present invention;

FIG. 14 illustrates a perspective view of an alternative collet member of an endoscopic hand instrument, in accordance with a fifteenth embodiment of the present invention;

FIG. 15 illustrates an elevational view of an alternative collet member of an endoscopic hand instrument, in accordance with a sixteenth embodiment of the present invention;

FIG. 16 illustrates a sectional view taken along line II-II of FIG. 15, in accordance with a seventeenth embodiment of the present invention;

FIG. 17 illustrates a partial sectional view of a tip system engaged to an alternative collet member of an endoscopic hand instrument, in accordance with an eighteenth embodiment of the present invention;

FIG. 18 illustrates a partial sectional view of a tip system engaged to an alternative collet member of an endoscopic hand instrument, in accordance with an nineteenth embodiment of the present invention;

FIG. 19 illustrates an elevational view of a tip system about to be engaged to a collet member of an endoscopic hand instrument, in accordance with a twentieth embodiment of the present invention;

FIG. 20 illustrates a partial sectional view of a tip system about to be engaged to a collet member of an endoscopic hand instrument, in accordance with a twenty-first embodiment of the present invention;

FIG. 21 illustrates an elevational view of a tip system partially engaged to a collet member of an endoscopic hand instrument, in accordance with a twenty-second embodiment of the present invention;

FIG. 22 illustrates a partial sectional view of a tip system partially engaged to a collet member of an endoscopic hand instrument, in accordance with a twenty-third embodiment of the present invention;

FIG. 23 illustrates an elevational view of a tip system fully engaged to a collet member of an endoscopic hand instrument, in accordance with a twenty-fourth embodiment of the present invention;

FIG. 24 illustrates a partial sectional view of a tip system fully engaged to a collet member of an endoscopic hand instrument, in accordance with a twenty-fifth embodiment of the present invention;

FIG. 25 illustrates an exploded view of an alternative endoscopic hand instrument, in accordance with a twenty-sixth embodiment of the present invention;

FIG. 26 illustrates a partial sectional view of an alternative endoscopic hand instrument, in accordance with a twenty-seventh embodiment of the present invention;

FIG. 27 illustrates an elevational view of a tip system about to be engaged to an alternative endoscopic hand instrument, in accordance with a twenty-eighth embodiment of the present invention; and

FIG. 28 illustrates a partial sectional view of a tip system about to be engaged to an alternative endoscopic hand instrument, in accordance with a twenty-ninth embodiment of the present invention.

The same reference numerals refer to the same parts throughout the various Figures

### DETAILED DESCRIPTION OF THE INVENTION

The following description of the preferred embodiment(s) is merely exemplary in nature and is in no way intended to limit the invention, or uses. The use of the terms “distal,” “proximal,” “first,” “second,” and/or the like, are meant for references purposes only and are not meant to limit the scope of the present invention.

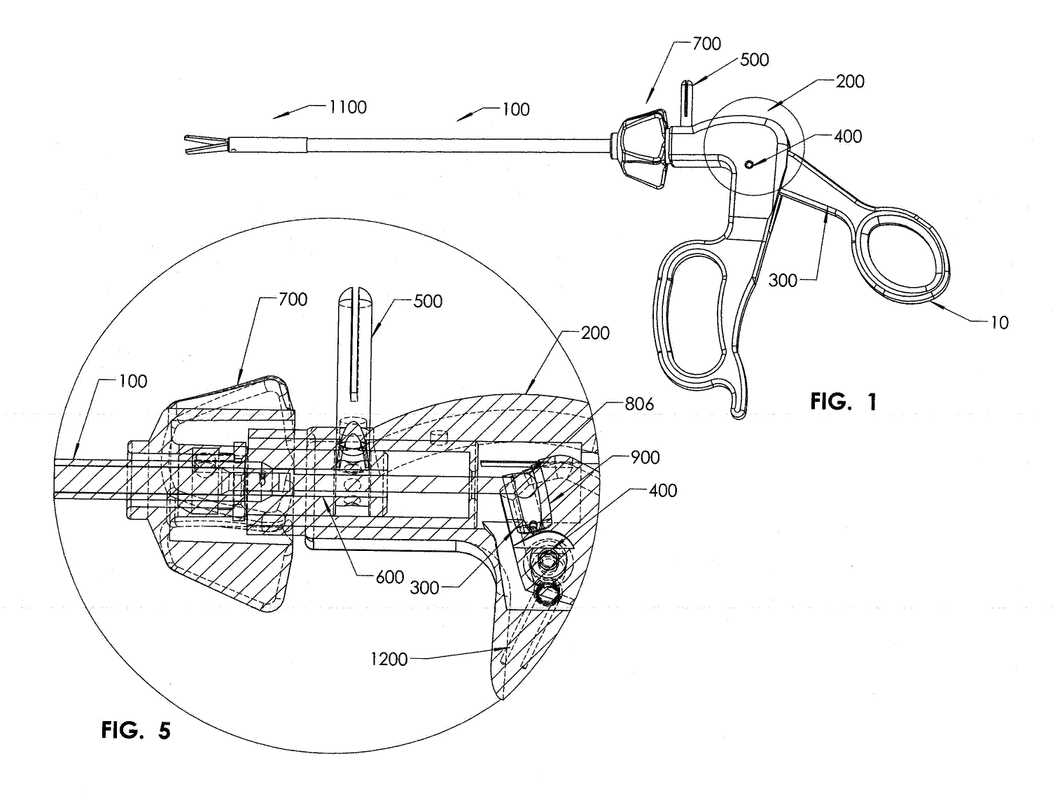

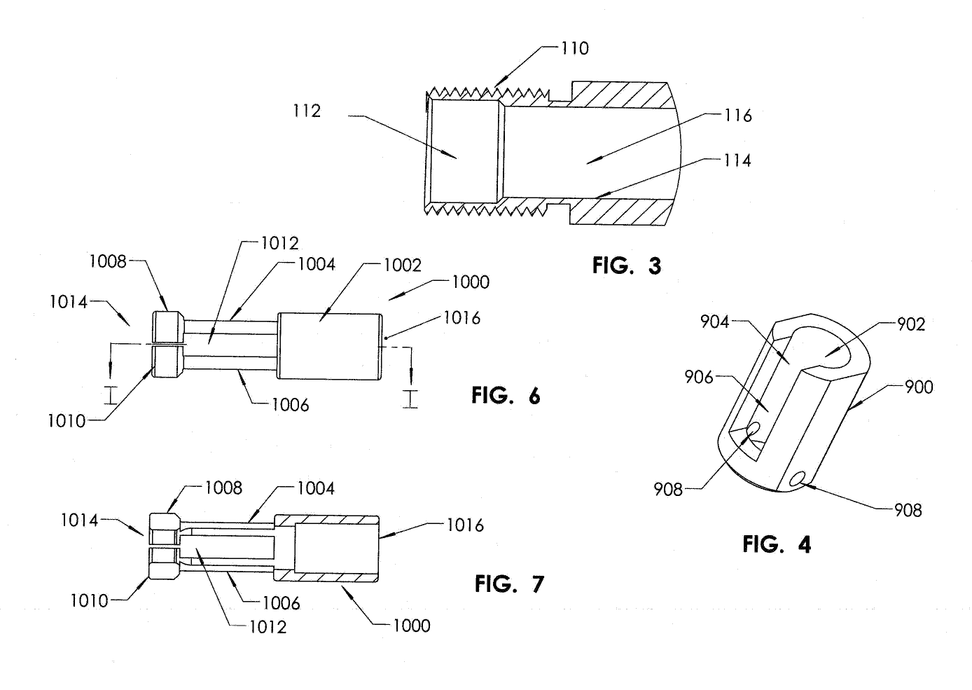

Referring to the drawings generally, and specifically to FIGS. 1-5, there is shown an endoscopic hand instrument generally at 10.

In accordance with one aspect of the present invention, a tube member 100 may be provided. The tube member 100 may include a substantially elongated cylindrical shape (although other shapes can be practiced with the present invention) having a first (e.g., distal) open end 102 and a spaced and opposed second (e.g., proximal) open end 104. An area defining a throughbore 106 may be provided, thus permitting communication between the first open end 102 and the second open end 104. At a distal portion 108 of the tube member 100, an external threaded portion 110 may be provided, e.g., in proximity to the first open end 102, the intended purpose of which will be described herein. The external threaded portion 110 of the distal portion 108 may be provided with a radially expanded inner diameter portion 112, e.g., relative to other portions of the inner diameter 114 of the throughbore 106. That is, the other portions of the inner diameter 114 of the throughbore 106 may be relatively smaller than that of the inner diameter portion 112.

In accordance with one aspect of the present invention, a stationary handle member 200 may be provided. By “stationary,” as that term is used herein, it is meant to include any handle member that remains substantially fixed relative to another movable handle member operably associated therewith. An area defining a bore 202 may be provided in a body portion 204 thereof, the intended purpose of which will be described herein.

By way of a non-limiting example, a proximal portion 112 of the tube member 100 may be operable to be at least partially received in the bore 202 of the stationary handle member 200. By way of another non-limiting example, the tube member 100, once partially received within the bore 202 of the stationary handle member 200, may be substantially freely rotatable thereabout.

In accordance with one aspect of the present invention, a movable handle member 300 may be provided. By “movable,” as that term is used herein, it is meant to include any handle member that can move, pivot or rotate relative to another stationary handle member operably associated therewith. The movable handle member 300 may be pivotable on the stationary handle member 200 via a fastener 400 (e.g., a pin, bolt, and/or the like). In this manner, the movable handle member 300 may move in a pivoting or rotating action relative to the stationary handle member 200, the intended purpose of which will be described herein.

In accordance with one aspect of the present invention, a power plug 500 may be provided. By way of a non-limiting example, the power plug 500 may be employed to provide electrical power (e.g., from an electrical power source (not shown)) to one or more components of the endoscopic hand instrument 10. By way of another non-limiting example, a proximal portion of an adapter member 600 may be secured to the stationary handle member 200 via the power plug 500. For example, the power plug 500 may anchor the adaptor member 600 to a sleeve member 700 while allowing the adaptor member 600, and hence the proximal portion 112 of the tube member 100, to be rotated relative to the stationary handle member 200. For example, the proximal portion 112 of the tube member 100 may be securely attached (e.g., glued, welded, soldered and/or the like) to the adaptor member 600. Rotation may be accomplished by applying finger pressure to a rotation knob member 700, which may also be firmly attached to the adaptor member 600. In this manner, rotation of the knob member 700 causes the adapter member 600, as well as the tube member 100 (e.g., being fastened to the adapter member 600) to rotate thereabout.

In accordance with one aspect of the present invention, an actuation or activation shaft member 800 may be provided. By way of a non-limiting example, the activation shaft member 800 may be comprised of an elastic conductive material such as but not limited to stainless steel. By way of another non-limiting example, the activation shaft member 800 may include a first end (e.g., distal) portion 802 and a second spaced and opposed second end (e.g., proximal) portion 804. The second end portion 804 may be formed as a ball-shaped member 806, the intended purpose of which will be described herein. By way of a non-limiting example, the activation shaft member 800 may be comprised of two elements, 808, 810, respectively, that may be joined at ends 812, 814, respectively (e.g., via a pin (not shown)).

Referring again to FIGS. 1-5, and in accordance with one aspect of the present invention, a bushing member 900 may be provided. By way of a non-limiting example, the bushing member 900 may be provided with an area defining a groove 902 formed therein. By way of another non-limiting example, the groove 902 may be provided with a first area 904 and a second area 906, wherein the first area 904 is sized to easily receive the ball-shaped member 806 and the second area 906 is sized so as to prevent the inadvertent dislodgement of the ball-shaped member 806. By way of still another non-limiting example, the bushing member 900 may be positioned in the bore 302 of the body portion 304 of the movable handle member 300. In this manner, the ball-shaped member 806 may be removably secured to the movable handle member 300 via the bushing member 900, thus permitting engagement of the activation shaft member 800 to the movable handle member 300. By way of a non-limiting example, the ball-shaped member 806 may be initially received in the first area 904 and substantially prevented from being dislodged therefrom by the second area 906. By way of a non-limiting example, the activation shaft member 800 may be substantially free to move reciprocally (e.g., in a “to and fro” and/or “back and forth” manner) inside the tube member 100, e.g., when activated by the movable handle member 300.

Optionally, the bushing member 900 may be removably secured to the movable handle member 300 via a pin and/or the like (not shown) that can be situated in one or more holes 908 formed therein, e.g., to prevent or restrain relative movement of the bushing member 900. The bushing member 900 may also be secured to the movable handle member 300 by the same pin (not shown) through pin hole 909.

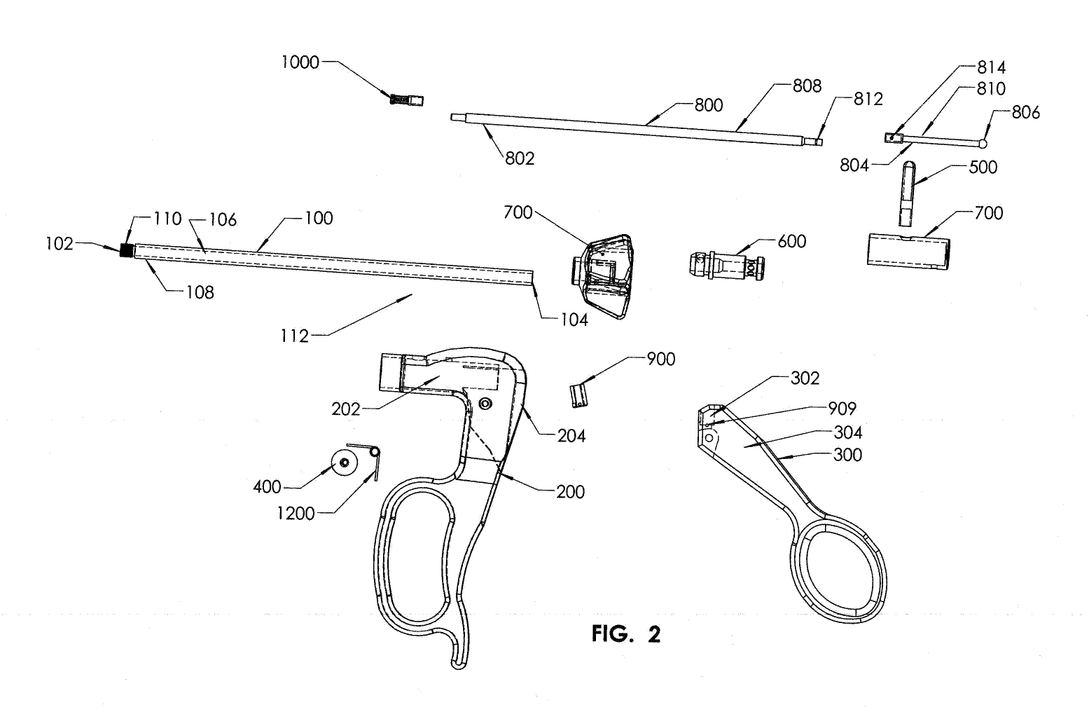

Referring specifically to FIGS. 2 and 6-9, and in accordance with one aspect of the present invention, a collet member 1000 may be provided. By way of a non-limiting example, the collet member 1000 may be comprised of an elastic conductive material such as but not limited to stainless steel. The collet member 1000 may include a body portion 1002 and at least two arm members 1004, 1006, respectively, extending therefrom. It should be appreciated that more than two arm members may be used in the practice of the present invention. The two arm members 1004, 1006, respectively, may be sufficiently biasable and/or flexible so as to be operable to flex in an outward and/or inward direction relative to a central axis of the collet member 1000. Each of the arm members 1004, 1006, respectively, may include end portions 1008, 1010, respectively, wherein the end portions 1008, 1010, respectively, may include an external diameter greater than the external diameter of the arm members 1004, 1006, respectively. An area defining a throughbore 1012 extends from a first end 1014 to a second end 1016 of the collet member 1000. The throughbore 1012 may provide an enclosure area 1018 between the two arm members 1004, 1006, respectively, the intended purpose of which will be described herein.

By way of another non-limiting example, the collet member 1000 may be firmly attached to the first end portion 802 of the activation shaft member 800.

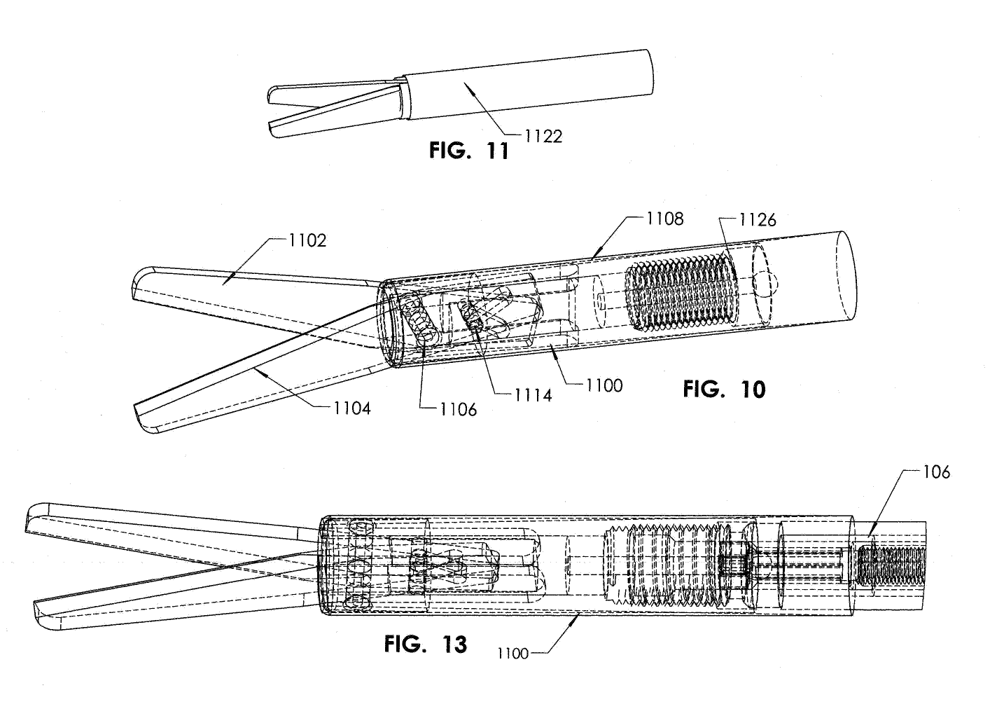

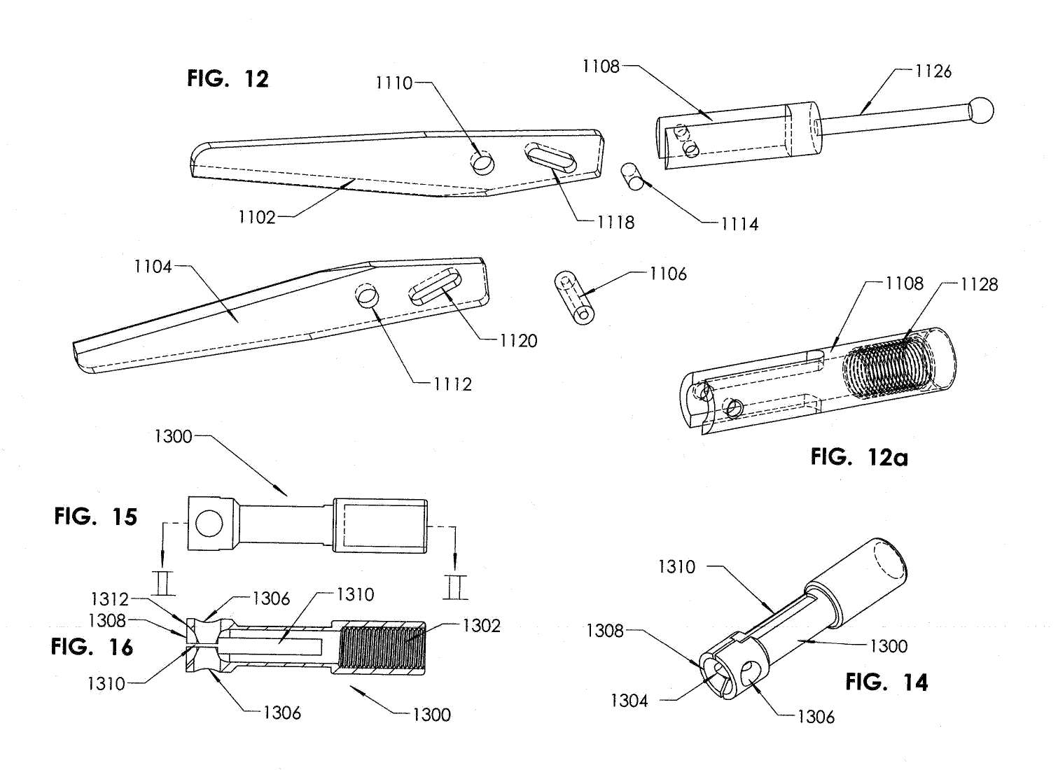

Referring specifically to FIGS. 10-13, and in accordance with one aspect of the present invention, a selectively attachable and/or detachable tip assembly 1100 may be comprised of a pair of jaw members 1102, 1104, respectively, both pivotable on a common pin member 1106, which may be secured to a clevis member 1108 and mounted through holes 1110, 1112, respectively, formed in the jaw members 1102, 1104, respectively. It should be appreciated that the jaw members 1102, 1104, respectively, may be configured to perform any number of functions, including but not limited to those typically performed by scissors, graspers, dissectors and/or the like. A pin member 1114 may be secured to a clevis member 1116 and mounted through cams 1118, 1120, respectively. The pivoting action may be accomplished by reciprocal movement of the clevis member 1108 and the attached pin member 1114. The common pin member 1106 may be secured to the clevis member 1108. The clevis member 1108 may be further covered with a non-conductive sleeve member 1122. The clevis member 1108 may include a ball-shaped member 1124 formed on a proximal end 1126 thereof, the intended purpose of which will be described herein. By way of a non-limiting example, the enclosure area 1018, together with the activating shaft member 800, when engaged to the collet member 1000, may form a cavity that can secure the ball-shaped member 1124 of the activating shaft member 800.

By way of a non-limiting example, when the tip assembly 1100 is engaged to the collet member 1000, the ball-shaped member 1124 may be simultaneously engaged to the enclosure area 1018 so as to removably fasten the tip assembly 1100 to the collet member 1000, and thus, the activation shaft member 800 as well.

In this manner, rotation of the knob member 700 also may cause rotation of the tip assembly 1100, e.g., for preferred alignment of the instrument by the surgeon.

Referring again to FIGS. 2-5, when no tip assembly is attached to the collet member, the activation shaft member 800 is pushed distally, e.g., by the counter clockwise (CCW) rotation of the movable handle member 300, e.g., caused by a torsion spring member 1200. This distal movement brings the arm members 1004, 1006, respectively, of the collet member 1000 within the expanded inner diameter 112 of the tube member 100, thus allowing the arm members 1004, 1006, respectively, to flex radially outward when the ball-shaped member 1124 is pushed towards the tube end of the handle assembly of the endoscopic hand instrument 10.

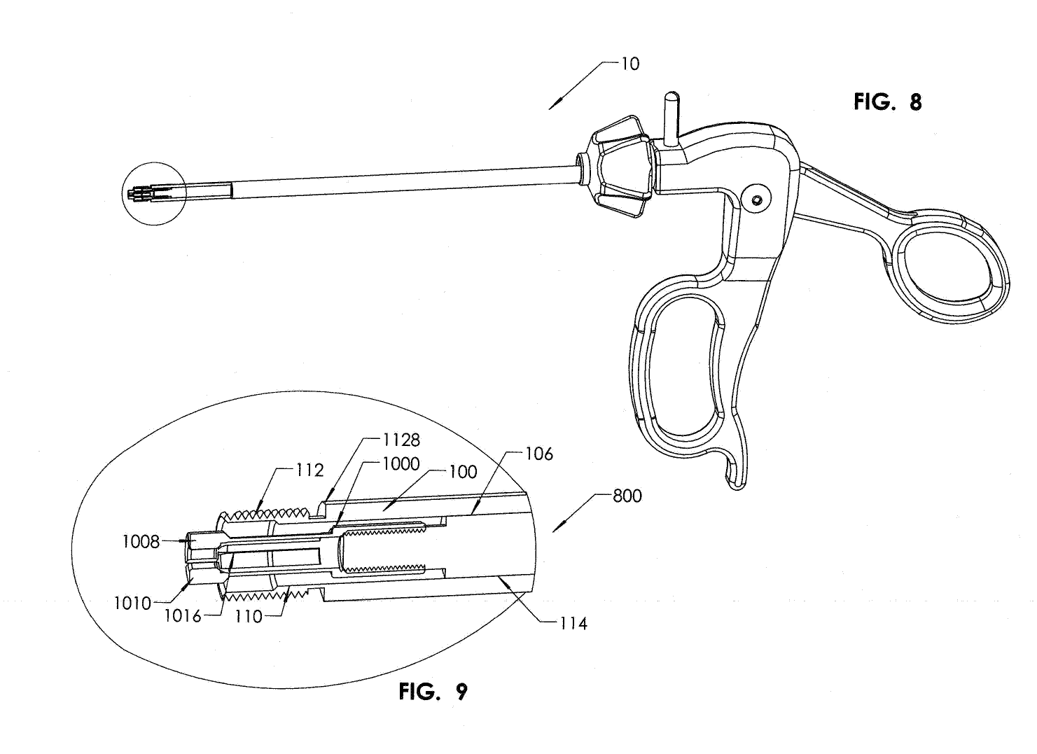

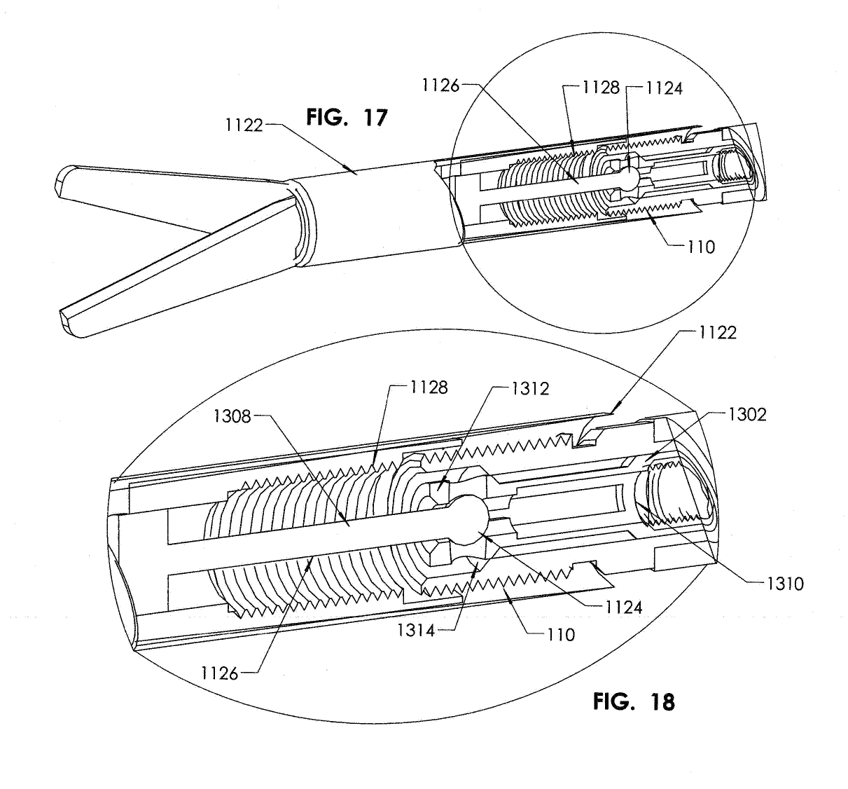

Referring to FIGS. 8-10, 13 and 17-18, the movable handle member 300 is normally in a fully open position, e.g., as a result of the spreading action by the torsion spring 1200, as long as no tip assembly is attached to the handle assembly of the endoscopic hand instrument 10. When a tip assembly is not attached, the movable handle member 300 may be spread open by the torsion spring 1200 and thus, the distal end of the collet member 1000 protrudes into the expanded inner diameter 112 of the tube member 100. As previously noted, the collet member 1000 may be made from an elastic conductive material, such as but not limited to stainless steel. This allows for the spreading apart of the arm members 1004, 1006, respectively, of the collet member 1000 within the expanded inner diameter 112 of the tube member 100. The radial spread of the arm members 1004, 1006, respectively, allows for the entrance of the ball-shaped member 1124 of the detachable tip assembly 1100 into the enclosure area 1012, prior to the collet member 1000 receding proximally into the smaller inner diameter 114 of the tube member 100, being proximal to inner diameter portion 112 and hence locking/securing the ball-shaped member 1124 within the collet member 1000. While the collet member 1000 is within the smaller inner diameter 114, the arm members 1004, 1006, respectively, cannot spread apart and release ball-shaped member 1124.

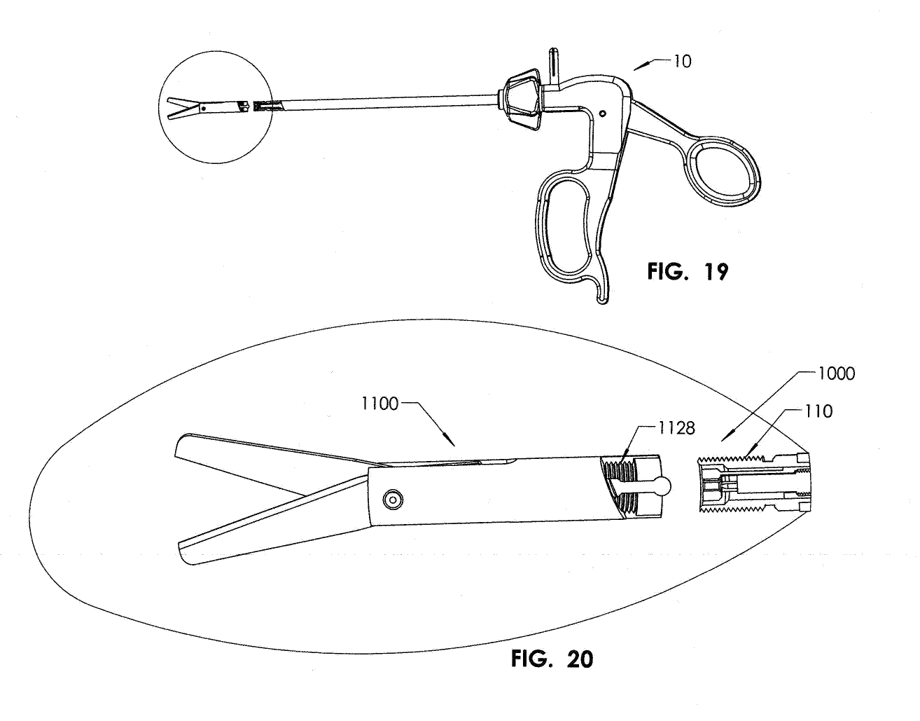

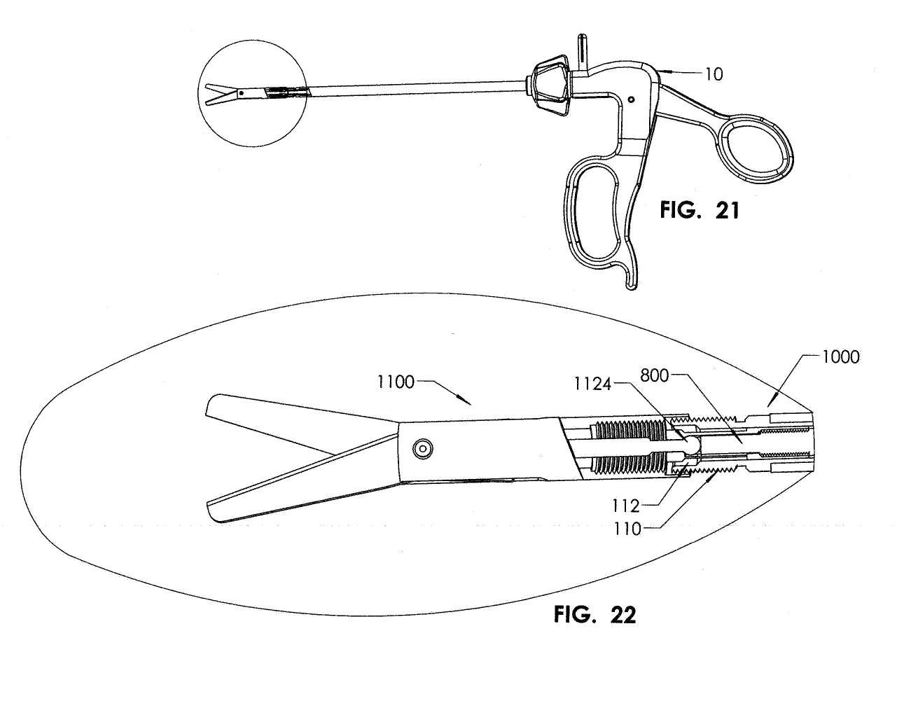

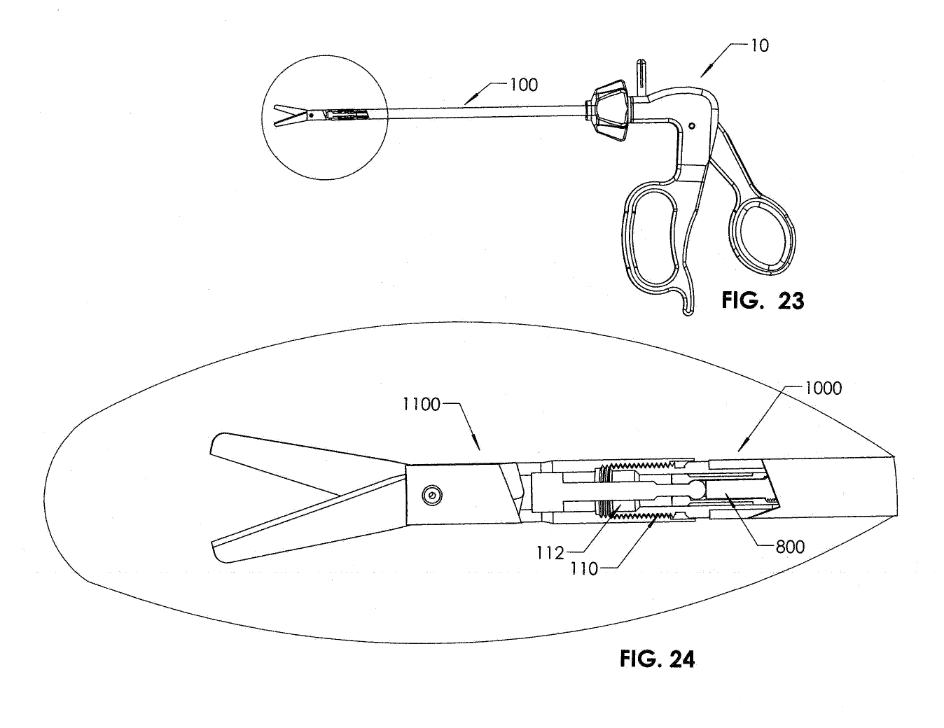

Referring to FIGS. 19-24, one can easily understand the manner in which one may attach and/or detach the tip assembly 1100 to the handle assembly of the endoscopic hand instrument 10.

When detachable tip assembly 1100 is pushed against the handle assembly of the endoscopic hand instrument 10, the ball-shaped member 1124 enters the collet member 1000 while spreading arm members 1004, 1006, respectively, aside, until resting within the enclosure area 1018. Continuation of the pushing movement of the tip assembly 1100 towards the handle assembly of the endoscopic hand instrument 10 results in an internal threaded portion 1128 of the tip assembly 1100 reaching the threaded portion 110 of the tube member 100. The user may then thread the tip assembly 1100 onto the tube member 100. As this process advances, the arm members 1004, 1006, respectively, of the collet member 1000 and the enclosure area 1012 are pushed deeper (i.e., proximally) within the tube member 100, eventually reaching confined zone 116 and thus eliminating the possible spreading apart action of the arm members 1004, 1006, respectively, thus securing the ball-shaped member 1124 to the activating shaft member 800. While the attachment of the tip assembly 1100 is occurring, the activating shaft member 800 is being forced rearwardly (i.e., proximally), wherein its ball-shaped member 806 is pushing the movable handle member 300 pivotally counterclockwise. Thus, the remaining range of movement of the activating shaft member 800 and the collet member 1000 (being securely attached to it) progresses while the collet member 1000 is securely grasping the ball-shaped member 1124. When the user spreads the handles apart, the activating shaft member 800 and the collet member 1000 are pushed distally and the jaw members are forced open by the advancement of the clevis member 1116. Hence, the activation of the reciprocal opening and closing of the jaw members 1102, 1104, respectively, is achieved via the fore and aft movement of the clevis member 1116, e.g., as a function of the movement of the movable handle member 300.

Detaching the tip assembly 1100 simply requires the unthreading of the tip assembly 1100 off of the tube member 100 and a relatively light pull of the tip assembly 1100 away from the collet member 1000.

Referring specifically to FIGS. 14-16 and 19-24, an alternative collet member 1300 is shown. By way of a non-limiting example, the collet member 1300 may include a threaded portion 1302, allowing for its attachment to an activating shaft member 1400. The collet member 1300 may include an area defining axial hole 1304 and perpendicular hole 1306. A leading taper 1308, as well as a shaped slot 1310 that creates partial arm members 1312, 1314, respectively, allowing them to elastically spread radially upon insertion of a ball-shaped member 1500 may be provided. Upon insertion, the ball-shaped member 1500 spreads the arm members 1312, 1314, respectively, and comes into position within axial hole 1304 and perpendicular hole 1306. Both collet members 800 and 1300 are operable to be advanced distally, e.g., by their respective activating shaft members, and movable handle member 300. Thus, by properly selecting the torsion spring member 1200 and the angular position of the bushing member 900, the collet members 800, 1300, respectively, will accept the ball-shaped member 1124, 1500, respectively, into the collet members 800, 1300, respectively, without rescission into the confined zone 116 in the tube member 100. The collet members 800, 1300, respectively, will recede upon continuous pushing by the user, forcing the threaded portion 1128 of the tip assembly 1100 to engage with the threaded portion 110 of the tube member 100.

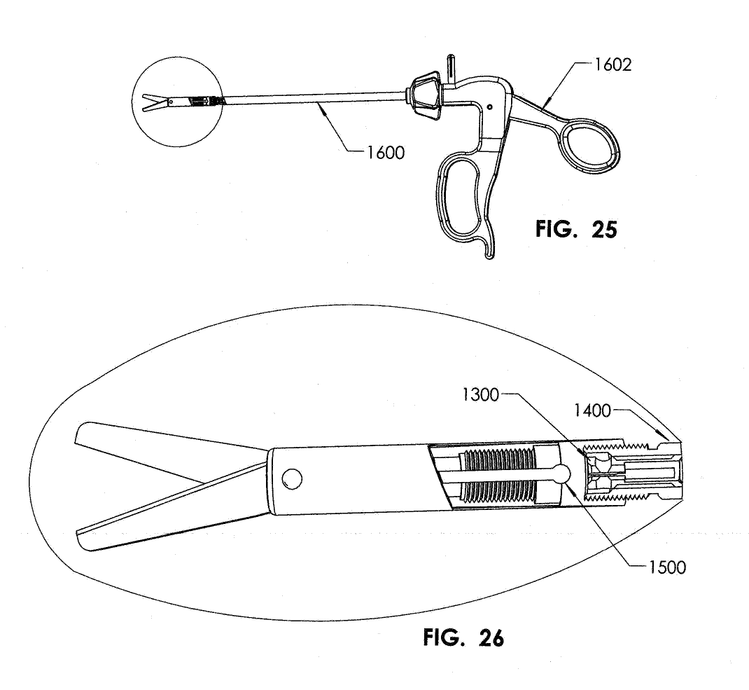

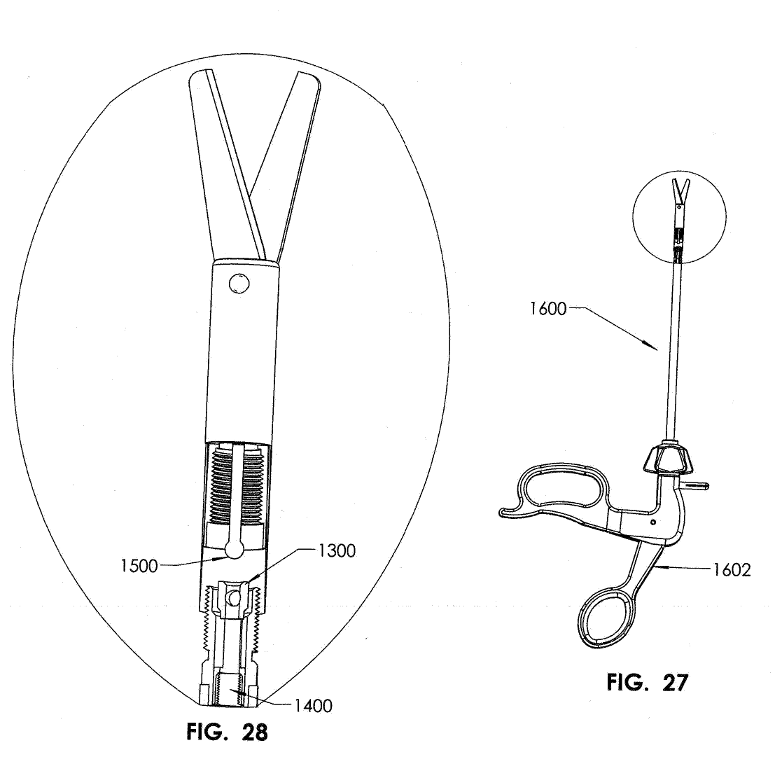

Referring specifically to FIGS. 25-28, there is shown an alternative endoscopic hand instrument generally at 1600. In this embodiment, the movable handle member 1602 is relatively heavy and loose enough to be spread opened once the handle assembly of the endoscopic hand instrument 1600 is held in a vertical position (e.g., see FIGS. 27-28). This design thus eliminates the need for a torsion spring member and may found to be preferred by some users. It should be appreciated that both collet members 1000, 1300, respectively, can be practiced with this particular embodiment.

In summary, some of the advancements as offered by the present invention are, without limitation: (1) the automatic advancement of the distal arm members of the collet member beyond the tube member's smaller internal diameter portion into the expanded diameter portion, so that the collet member may readily accept the tip assembly's clevis ball-shaped member, which then becomes secured within the collet member's arm members once pushed proximally by the user, e.g., when threading the tip assembly onto the tube member's threaded portion; (2) similarly, the automatic advancement of the collet member, mounted on the activation shaft member and forced forward by the handle's torsion spring member, (thus leaving collet member in a position that allows the spreading of the arm members located distally), allows the ball-shaped member to enter the collet member and become secured therein once the user forces the tip assembly towards the tube member's threaded end to thread the tip assembly securely onto the handle assembly of the endoscopic hand instrument; and (3) the inclusion of the last variant, e.g., as shown in FIGS. 25-28, wherein the movable handle member, spread by its own weight when held vertically, thus advancing the collet member distally so that attachment of the tip assembly could be executed without the need for a torsion spring member.

While the invention has been described with reference to an exemplary embodiment, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An endoscopic hand instrument, comprising:
a tubular member having an area defining a throughbore extending between a first open end and a second open end, wherein the throughbore includes a first portion having a first inner diameter and a second portion having a second inner diameter, wherein the first inner diameter is greater than the second inner diameter;
a collet member selectively operable to be at least partially received within the throughbore, wherein the collet member includes at least two biasable arm members, wherein the collet member is biased such that the two arm members extend radially outward into the first portion of the throughbore, wherein the two arm members include an area defining an aperture; and
a tip assembly having an engagement member, wherein the engagement member is selectively operable to be received between the two arm members and into the aperture, wherein the tip assembly is selectively operable to engage the tubular member so as to force the two arm members into the second portion of the throughbore such that the two arm members are prevented from extending radially outward whereby the engagement member is secured in the aperture.

2. The invention according to claim 1, further comprising:
an actuation shaft member selectively operable to be at least partially received within the throughbore, wherein the actuation shaft member is selectively operable to more in a to and fro direction within the throughbore.

3. The invention according to claim 2, wherein the collet member is operably associated with an end portion of the actuation shaft member.

4. The invention according to claim 2, further comprising:
a handle assembly operably associated with the tubular member, wherein the handle assembly includes at least one movable handle member.

5. The invention according to claim 4, wherein at least one end of the actuation shaft member is operably associated with the movable handle member.

6. The invention according to claim 2, wherein the tip assembly includes at least two jaw members.

7. The invention according to claim 6, wherein the to and fro movement of the actuation shaft member is selectively operable to open and close the jaw members.

8. The invention according to claim 5, wherein the tip assembly includes at least two jaw members.

9. The invention according to claim 8, wherein the movable handle member is selectively operable to open and close the jaw members.

10. An endoscopic hand instrument, comprising:
a tubular member having an area defining a throughbore extending between a first open end and a second open end, wherein the throughbore includes a first portion having a first inner diameter and a second portion having a second inner diameter, wherein the first inner diameter is greater than the second inner diameter;
an actuation shaft member selectively operable to be at least partially received within the throughbore, wherein the actuation shaft member is selectively operable to more in a to and fro direction within the throughbore;
a collet member operably associated with an end portion of the actuation shaft member, wherein the collet member is selectively operable to be at least partially received within the throughbore, wherein the collet member includes at least two biasable arm members, wherein the collet member is biased such that the two arm members extend radially outward into the first portion of the throughbore, wherein the two arm members include an area defining an aperture; and
a tip assembly having an engagement member, wherein the engagement member is selectively operable to be received between the two arm members and into the aperture, wherein the tip assembly is selectively operable to engage the tubular member so as to force the two arm members into the second portion of the throughbore such that the two arm members are prevented from extending radially outward whereby the engagement member is secured in the aperture.

11. The invention according to claim 10, further comprising:
a handle assembly operably associated with the tubular member, wherein the handle assembly includes at least one movable handle member.

12. The invention according to claim 11, wherein at least one end of the actuation shaft member is operably associated with the movable handle member.

13. The invention according to claim 10, wherein the tip assembly includes at least two jaw members.

14. The invention according to claim 13, wherein the to and fro movement of the actuation shaft member is selectively operable to open and close the jaw members.

15. The invention according to claim 12, wherein the tip assembly includes at least two jaw members.

16. The invention according to claim 15, wherein the movable handle member is selectively operable to open and close the jaw members.

17. An endoscopic hand instrument, comprising:
a tubular member having an area defining a throughbore extending between a first open end and a second open end, wherein the throughbore includes a first portion having a first inner diameter and a second portion having a second inner diameter, wherein the first inner diameter is greater than the second inner diameter;
a handle assembly operably associated with the tubular member, wherein the handle assembly includes at least one movable handle member;
an actuation shaft member selectively operable to be at least partially received within the throughbore, wherein at least one end of the actuation shaft member is operably associated with the movable handle member, wherein the actuation shaft member is selectively operable to more in a to and fro direction within the throughbore;
a collet member operably associated with an end portion of the actuation shaft member, wherein the collet member is selectively operable to be at least partially received within the throughbore, wherein the collet member includes at least two biasable arm members, wherein the collet member is biased such that the two arm members extend radially outward into the first portion of the throughbore, wherein the two arm members include an area defining an aperture; and
a tip assembly having an engagement member, wherein the engagement member is selectively operable to be received between the two arm members and into the aperture, wherein the tip assembly is selectively operable to engage the tubular member so as to force the two arm members into the second portion of the throughbore such that the two arm members are prevented from extending radially outward whereby the engagement member is secured in the aperture.

18. The invention according to claim 17, wherein the tip assembly includes at least two jaw members.

19. The invention according to claim 18, wherein the to and fro movement of the actuation shaft member is selectively operable to open and close the jaw members.

20. The invention according to claim 17, wherein the movable handle member is selectively operable to open and close the jaw members.
